(19) United States
(12) Patent Application Publication
(10) Pub. No.: US 2023/0047439 A1
(43) Pub. Date: Feb. 16, 2023

(54) IMPURITY FORMATION REDUCTION DURING PRODUCT REFINING

(71) Applicant: INV NYLON CHEMICALS AMERICAS, LLC, Wilmington, DE (US)

(72) Inventors: Benjamin David HERZOG, Wichita, KS (US); Milind V. KANTAK, Wilmington, DE (US); William J. TENN, III, Beaumont, TX (US)

(21) Appl. No.: 17/793,940

(22) PCT Filed: Feb. 1, 2021
(86) PCT No.: PCT/IB2021/050784

§ 371 Date: Jul. 20, 2022

Related U.S. Application Data
(60) Provisional application No. 62/968,169, filed on Jan. 31, 2020.

(51) Int. Cl. C07C 253/34

(52) U.S. Cl. CPC C07C 253/34

(57) ABSTRACT

Disclosed is a method for purifying a crude adiponitrile stream by differential volatility comprising separating at least a portion of the components of the crude adiponitrile stream by flashing vapor from a liquid film.

### FIELD

The present application relates to the field of reduction of undesired impurity formation during product refining.

### BACKGROUND

Dinitrile compounds, such as adiponitrile (ADN), are important commercial chemicals. The most important application of dinitriles being as intermediates in the production of diamine monomers, which are useful in the synthesis of various polyamide polymers. The hydrogenation of ADN provides hexamethylenediamine (HMD), which is one of the essential ingredients used to manufacture nylon-6,6 (N66) and other nylons e.g. nylon-6,10 and nylon-6,12. N66 is produced by reacting HMD with adipic acid (AA) to form an aqueous salt solution. The N66 polymer can be used to produce synthetic fibers and engineering polymers which are of great commercial value.

Formation of the impurity 2-cyanocyclopentylideneimine (CPI) by cyclization of ADN occurs during refining of crude ADN. Hydrogenation of the CPI during production of HMD results in 2-aminomethylcyclopentylamine (AMC), which adversely impacts the quality of N66 formed from polymerization of HMD with AA.

The removal of CPI from ADN before the hydrogenation step is difficult, as the relative volatility of CPI to ADN is 1.45. However, the removal of AMC [CPI upon hydrogenation] from HMD is much more difficult, as the relative volatility of AMC to HMD is 1.20. Thus, to maintain low levels of AMC in the HMD product, it is most desirable to remove the CPI from the ADN prior to hydrogenation. The removal of CPI from ADN by distillation and other techniques is very difficult. The primary method for removing CPI from ADN is vacuum distillation, but when sufficiently low levels of CPI cannot be achieved, other options have been employed to remove the CPI after distillation.

US 3,496,212 relates to the use of a water-soluble aldehyde in combination with an extraction step using an aromatic solvent and water followed by an additional distillation step.

Canadian patent 672,712 relates to ozone treatment of ADN to destroy CPI.

US 3,758,545 relates to the use of paraformaldehyde to chemically react with CPI.

US 3,775,258 relates to a method for hydrolyzing CPI to a ketone using an acidic catalyst at 140° C.

Canadian patent 1,043,813 uses a weak cation exchange resin to remove CPI from ADN.

Thus one approach to providing a refined ADN product that is relatively depleted in CPI is to remove CPI from crude ADN. One problem with this approach is that removing the CPI may trigger the formation of additional CPI, provided that the ADN is exposed to suitable conversion conditions.

From a yield perspective, it would be desirable to prevent the formation of the CPI during the ADN manufacturing process. U.S. 2018/0244607 A1 teaches a process for inhibiting the formation of CPI by the use of a Brønsted acid to suppress its formation. It would be desirable to provide a process that does not require the introduction of additional components into the crude or refined ADN stream.

There is therefore a need in the art for processes for the preparation of dintriles, such as ADN, of high purity in which the formation of undesirable, difficult to separate byproducts such as CPI is reduced.

In order to produce high quality N66 polymer, the essential ingredients such as HMD must be of extremely high purity. The presence of impurities such as AMC can cause undesirable effects in polymer end-products. Undesirable impurities in the HMD are removed primarily by distillation operations, while impurities in the AA are removed primarily by crystallization. Commercially produced HMD generally contains two kinds of impurities: those produced during hydrogenation of ADN; and those resulting from the reaction of impurities that are contained in the feed ADN. One of the most detrimental impurities to N66 quality is 2- AMC, which is formed primarily by the hydrogenation of the impurity CPI which is present in the ADN.

One method producing ADN is the hydrocyanation of 1,3-butadiene to 3-pentenenitrile (3PN), followed by the hydrocyanation of the 3PN to ADN. These hydrocyanations are performed using a nickel (0) catalyst stabilized with phosphite ligands. These phosphite ligands can be either monodentate and/or bidentate ligands. The hydrocyanation of 3PN also requires the use of a Lewis acid co-catalyst. When using a monodentate or bidentate ligand catalyst system, zinc chloride is a suitable Lewis acid. One of the advantages of the direct hydrocyanation process is the hydrocyanation of 3PN takes place at mild temperature conditions where virtually no CPI is generated. However, in order to refine the crude ADN to the high purity product required for hydrogenation to HMD, several distillation steps are required. Because of the very low vapor pressure of ADN, these distillations involve temperatures as high as 200° C. At these high temperatures, CPI generation can occur during these distillation operations.

Impurities in the HMD are removed by distillation in commercial production facilities. The removal of AMC to meet HMD quality specifications can limit the capacity of a commercial production facility resulting in lost production and sales. Therefore, minimizing the CPI level in the ADN feed to an HMD production facility has significant economic value.

### SUMMARY

Disclosed is a method for reducing formation of CPI from ADN. It has been found that the addition of a falling-film evaporator to the distillation unit for purification of the crude dinitriles retards the formation of CPI. Disclosed is a method for purifying a crude adiponitrile stream by differential volatility comprising separating at least a portion of the components of the crude ADN stream by flashing vapor from a liquid film.

In the disclosed method, the liquid film can flow downwardly on a substantially vertical wall. A falling film evaporator is disclosed in U.S. Pat. 4,918,944 to Takahashi et al. (Hitachi).

A method and apparatus for short-path distillation is disclosed is U.S. Pat. 4,517,057 to Fauser et al. (Leybold AG).

The heat of vaporization for flashing vapor can be at least partially drawn from the sensible heat of the liquid film.

CPI can be a component of the crude ADN stream.

The step of flashing vapor from a liquid film can be followed by multistage distillation.

The step of flashing vapor from a liquid film can be preceded by multistage distillation. The multistage distillation can be carried out under at least partial vacuum.

The step of flashing vapor from a liquid film can be carried out under at least partial vacuum.

The method can further comprise controlling the temperature of the liquid film to reduce formation of CPI.

The method can include flashing from the liquid film at any suitable combination of temperatures and pressures. One example of such conditions includes:
a. Temperature of from 160° C. to 220° C.; and
b. Pressure of from 0.3 psia to 0.6 psia.

For example, it can include flashing at temperature from 175° C. to 205° C.; and pressure of from 0.35 psia to 0.5 psia. The flashing conditions can more specifically include temperature of from 180° C. to 200° C.; and pressure of from 0.4 psia to 0.45 psia.

The method can include flashing a minor amount of vapor from liquid held on a horizontal surface, for example, from ≥0% to less than 20% by weight of liquid flashed, for example, from ≥0% to ≤10% by weight of liquid flashed, for example from ≥0% to ≤5% by weight of liquid flashed.

### BRIEF DESCRIPTION OF THE FIGURES

FIG. 1 is a schematic representation of an embodiment 100 according to Example 1.

FIG. 2 is a schematic representation of an embodiment 200 according to Example 3.

FIG. 3 is a schematic representation of an embodiment 300 according to Example 5.

The following Examples demonstrate the present invention and its capability for use. The invention is capable of other and different embodiments, and its several details are capable of modifications in various apparent respects, without departing from the spirit and scope of the present invention. Accordingly, the Examples are to be regarded as illustrative in nature and nonlimiting. All parts and percentages are by weight unless otherwise indicated.

### EXAMPLE 1

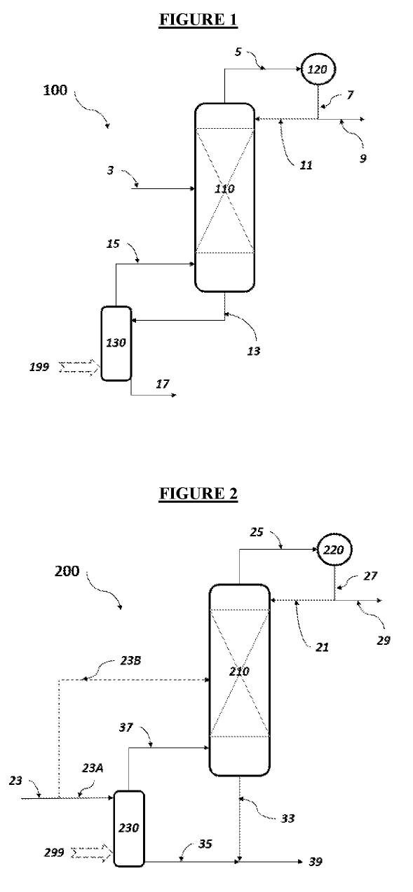

FIG. 1 is a schematic representation of an embodiment labeled 100 according to the present disclosure. Referring to FIG. 1, a Falling Film Evaporator (FFE) Unit 130 was added to the distillative operation utilizing a distillation Unit 110 and an overhead condenser Unit 120. Distillation Unit 110 was used for producing a refined ADN material stream 9. Unit 110 may be a packed, structured packing, trayed or some combination of the two vapor-liquid contacting methods.

A crude ADN stream 3 containing by-product components was fed to Unit 110 and refined to obtain high-purity ADN product. The vaporous stream 5 from Unit 110 was fed to the overhead condenser Unit 120 which condensed it to a condensate liquid stream 7. The condensate stream 7 may be split to provide a liquid reflux feed stream 11 to the top section of Unit 110. The remaining condensate, i.e., refined stream 9 was directed to the product storage and further use. The distillation unit 110 Tails (or bottoms) stream 13 was collected at the column Unit 110 bottom and directed to the FFE Unit 130. The Tails stream 13 is mostly a concentrated liquid stream containing high-boiling byproducts that were present in stream 3 entering the column Unit 110.

The FFE Unit 130 provides the non-horizontal wall surface over which the fed liquid stream 13 is distributed and forms a thin film adhering to its internal wall surface. The liquid stream 13 flowrate is such adjusted that a uniform liquid film forms, wets the internal wall surfaces and downflows in Unit 130. A heat input stream 199 was supplied to Unit 130 that provided the necessary energy for generating the vapors from the falling liquid film. The FFE Unit 130 may employ internally mounted rotating blades or wipers to aid in evenly spreading the falling liquid film over the wall surfaces.

The FFE Unit 130 vaporized part of the tails stream 13 and the up-flowing vapor stream 15 was returned to the column Unit 110 below the bottom tray, or packed section. This example demonstrates how the FFE Unit 130 surprisingly allowed high tails flow (14,000 lb/hr of stream 13) from the bottom of the column Unit 110. The high tails flow out of the column Unit 110 base reduced hold-up time for the materials exposed to the column base conditions, and also, reduced zinc chloride concentration in the base of column Unit 110. Zinc chloride is an impurity entering through the crude ADN stream 3. Reduced formation of CPI was observed that resulted in the reduced levels of CPI in the refined ADN stream 9 overhead.

Table 1 below provides the stream composition data according to FIG. 1 and present Example 1.

Table 1

Stream Balance

| | | Column Feed (Stream 3) | Column Make (Stream 9) | FFE Feed (Stream 13) | FFE bottoms (Stream 17) | FFE vapors (Stream 15) |
|---|---|---|---|---|---|---|
| Temperature | °C | 203.4 | 125.0 | 175.8 | 176.3 | 176.3 |
| Pressure | Psia | 1.26 | 0.23 | 0.44 | 0.44 | 0.44 |
| Mass Enthalpy | Btu/lb | 477.70 | 401.85 | 440.66 | 426.29 | 691.79 |
| Mass Density | 1b/ft³ | 45.84 | 55.12 | 52.90 | 53.26 | 0.01 |
| Mass Flows | lb/hr | 90,500 | 90,230 | 14,000 | 5,977 | 8,023 |
| Mass Fractions | | | | | | |
| Dinitriles | Wt.% | 99.70 | 99.84 | 98.96 | 97.67 | 99.92 |
| Metals | Wt.% | 0.099 | 0.000 | 0.637 | 1.493 | 0.0 |
| CPI | Wt.% | 0.025 | 0.042 | 0.020 | 0.016 | 0.025 |
| Others | Wt.% | 0.174 | 0.121 | 0.382 | 0.823 | 0.054 |

Table 2

CPI Component Balance

| | | Column Feed (Stream 3) | Column Make (Stream 9) | FFE Feed (Stream 13) | FFE bottoms (Stream 17) | FFE vapors (Stream 15) |
|---|---|---|---|---|---|---|
| CPI | lb/hr | 22.6 | 37.9 | 2.80 | 0.96 | 2.01 |

Net CPI formation in Unit 110 operation and according to Example 1 = (37.9 + 0.96) -(22.6) = 16.3 lb/hr. The CPI level in the refined ADN stream 9 was 0.042 wt.% (or 420 ppm by weight) of the total weight.

### Comparative Example 2

The above distillative process, described in Example 1 (and FIG. 1), was run except the FFE Unit 130 was not employed for processing the column Unit 110 Tails stream 13. The column Unit 110 boil-up energy was supplied to the column base using a conventional reboiler arrangement. This resulted in a lower tails stream 13 flow (6000 lb/hr) from the base of the column Unit 110 compared to that in Example 1 operation (14,000 lb/hr).

Table 3 below provides the stream composition data according to FIG. 1 and present Example.

Table 3

Stream Balance

| | | Column Feed (Stream 3) | Column Make (Stream 9) | Column Tails (Stream 13) |
|---|---|---|---|---|
| Temperature | °C | 203.4 | 125.0 | 176.1 |
| Pressure | Psia | 1.26 | 0.23 | 0.44 |
| Mass Enthalpy | Btu/lb | 477.70 | 401.72 | 426.12 |
| Mass Density | 1b/ft³ | 45.84 | 55.12 | 53.25 |
| Mass Flows | lb/hr | 90,500 | 90,229 | 6,000 |
| Mass Fractions | | | | |
| Dinitriles | Wt.% | 99.70 | 99.81 | 97.67 |
| Metals | Wt.% | 0.099 | 0.000 | 1.487 |
| CPI | Wt.% | 0.025 | 0.065 | 0.031 |
| Others | Wt.% | 0.174 | 0.121 | 0.811 |

Table 4

CPI Component Balance

| | | Column Feed (Stream 3) | Column Make (Stream 9) | Column Tails (Stream 13) |
|---|---|---|---|---|
| CPI | lb/hr | 22.6 | 58.6 | 1.9 |

Net CPI formation in Unit 110 operation and according to Comparative Example 2 = (58.6 + 1.9) - (22.6) = 37.9 lb/hr. The CPI level in the refined ADN stream 9 was 0.065 wt.% (or 650 ppm by weight) of the total weight.

For the same crude feed processing rate, the use of FFE Unit 130 in Example 1 unexpectedly reduced the net CPI formation by more than twice (16.3 vs. 37.9 lb/hr), and correspondingly, the CPI level in the refined stream reduced by one-third (420 vs. 650 ppm by weight) when compared to that in Comparative Example 2 operation without the FFE Unit 130.

### Example 3

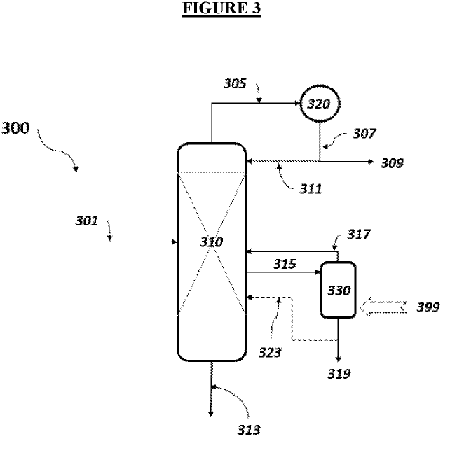

FIG. 2 is a schematic representation of an embodiment labeled 200 according to the present disclosure. Referring to FIG. 2, a Falling Film Evaporator (FFE) Unit 230 was added to the distillative operation utilizing a distillation Unit 210 and an overhead condenser Unit 220. Distillation Unit 210 was used for producing a refined adiponitrile material stream 29. Unit 210 may be a packed, trayed or some combination of the two vapor-liquid contacting methods.

A crude adiponitrile stream 23 containing by-product components was fed to FFE Unit 230 via stream 23A. Optionally, the total feed stream 23 may be split and a portion may be taken to the column Unit 210 via stream 23B (shown dotted). The vaporous stream 25 from Unit 210 was fed to the overhead condenser Unit 220 which condensed it to a condensate liquid stream 27. The condensate stream 27 may be split to provide a liquid reflux feed stream 21 to the top section of Unit 210. The remaining condensate, i.e., refined stream 29 was directed to the product storage and further use. The distillation unit 210 Tails (or bottoms) stream 33 was collected at the column Unit 210 bottom and combined with the tails stream 35 from the FFE Unit 230. The combined bottoms stream 39 was mostly concentrated in high-boiling byproducts that were present in stream 23.

The FFE Unit 230 was properly designed and sized to provide the non-horizontal wall surface over which the fed liquid stream 23 (or 23A) was distributed and formed a thin film adhering to its internal wall surface. The liquid stream 23 (or 23A) flowrate was such adjusted that a uniform liquid film formed, wetted the internal wall surfaces and flowed down the Unit 230. A heat input stream 299 was supplied to Unit 230 that provided the necessary energy for generating the vapors from the falling liquid film. The FFE Unit 230 may employ internally mounted rotating blades or wipers to aid in evenly spreading the falling liquid film over the wall surfaces.

The FFE Unit 230 vaporized part of the tails stream 23 (or 23A) and the up-flowing vapor stream 37 was returned to the column Unit 210 below the bottom tray, or packed section. This example demonstrates how the FFE Unit 230 surprisingly allowed high tails flow (14,000 lb/hr of stream 13) from the bottom of the column Unit 110. The high tails flow out of the column Unit 110 base reduced hold-up time for the materials exposed to the column base conditions, and also, reduced zinc chloride concentration in the base of column Unit 110. Reduced formation of CPI was observed that resulted in the reduced levels of CPI in the refined adiponitrile stream 9 overhead.

In this example (and FIG. 2) the tails stream 35 from the FFE Unit 230 containing most of the Zn components [for example, ZnCl₂ that enters with stream 23] was mixed with the column Unit 210 bottoms stream 33. This arrangement eliminated most of the Zn-containing components from accumulating in the column Unit 210 base. The column Unit 210 base would also have a higher hold-up time (of the order of minutes) than the residence time for the liquid in the FFE Unit 230 bottom (of the order of seconds). The significant lowering of the Zn level in the column base material in combination with the hold-up time reduction contributed to this surprising effect of overall CPI formation reduction.

The Example 3 operation may require more than one FFE Unit to handle the increased feed throughput. Such multiple FFE units can be designed, sized and flow connected using the conventional means. These may be cascading, parallel, series or any combination of such FFEs that are easy to operate and maintain.

Table 5 below provides the stream composition data according to FIG. 2 and present Example.

Table 5

Stream Balance

| | | FFE Feed (Stream 23 or 23A) | FFE Bottoms (Stream 35) | FFE Vapor (Stream 37) | Column Tails (Stream 33) |
|---|---|---|---|---|---|
| Temperature | °C | 51.0 | 202.6 | 159.7 | 199.0 |
| Pressure | Psia | 50.00 | 1.26 | 1.26 | 1.26 |
| Mass Enthalpy | Btu/lb | 295.94 | 477.04 | 511.87 | 473.45 |
| Mass Density | 1b/ft³ | 56.26 | 51.16 | 0.01 | 51.04 |
| Mass Flows | lb/hr | 118,000 | 81,638 | 36,362 | 8,950 |
| Mass Fractions | | | | | |
| Dinitriles | Wt.% | 76.52 | 99.68 | 24.52 | 99.63 |
| Metals | Wt.% | 0.076 | 0.109 | 0.000 | 0.000 |
| CPI | Wt.% | 0.000 | 0.000 | 0.000 | 0.000 |
| Others | Wt.% | 23.406 | 0.213 | 75.477 | 0.368 |

In this example [and according to the FIG. 2 arrangement and Table 5 data] no CPI formation is observed at the column base.

### Example 4

The data presented in Table 6 below show the effectiveness of incorporation of FFE unit into the design of a process for refining adiponitrile. The data showed that a distillative system integrated with the FFE unit reduced the CPI formation due to the Zn level reduction in the column base together with the reduced holdup time of the material accumulating at the column base.

Table 6

FFE Incorporation Effectiveness

| Zn Level in Dinitrile Column Base (ppm by wt.) | CPI Formation (lb/hr) | CPI in Dinitrile Product Stream (ppm by wt.) | FFE Feed (lb/hr) | FFE Vaporous Stream (lb/hr) |
|---|---|---|---|---|
| 5257 | 35.7 | 650 [Comp Ex. 2] | No FFE Unit | |
| 3154 | 21.1 | 487 | 5496 | 3981 |
| 2253 | 15.1 | 420 [Ex. 1] | 5458 | 8023 |
| 1752 | 11.7 | 382 | 5453 | 12028 |

Comparison of Example 1 (420 ppmw CPI in the refined product) with Comparative Example 2 (650 ppmw CPI in the refined product) showed significant reduction in both, the formation of CPI and the corresponding CPI level in the refined dinitrile (adiponitrile) product.

In addition, equipment fouling resulting from subsequent reactions of CPI was reduced as well. It was observed that the dinitrile distillation column calandrias routinely fouled from the CPI reactions/formation during dinitrile processing. Cleaning took on average 3-5 days and needed to be performed every 9-12 months of operation. The reduction of CPI formation according to the present disclosure greatly improved the equipment on-stream as a result of the reduced fouling.

### Example 5

FIG. 3 is a schematic representation of an embodiment labeled 300 according to the present disclosure. Referring to FIG. 3, a Falling Film Evaporator (FFE) Unit 330 is added to the distillative operation utilizing a distillation Unit 310 and an overhead condenser Unit 320. A liquid side-draw stream 315 from a suitable section of the distillation unit 310 is fed to the FFE Unit 330. Distillation Unit 310 is used for producing a refined adiponitrile material stream 309. Unit 310 may be a packed, trayed or some combination of the two vapor-liquid contacting methods.

A crude adiponitrile stream 301 containing by-product components is fed to Unit 310 and refined to obtain high-purity adiponitrile product. An overhead vaporous stream 305 from Unit 310 is fed to the overhead condenser Unit 320 which condenses the vapors to a condensate liquid stream 307. The condensate stream 307 may be split to provide a liquid reflux feed stream 311 to the top section of Unit 310. The remaining condensate, i.e., refined stream 309 is directed to the product storage and further use. The distillation unit 310 Tails (or bottoms) stream 313 is collected at the column Unit 310 bottom.

The FFE Unit 330 provides the non-horizontal wall surface over which the fed liquid side-draw stream 315 from Unit 310 is distributed and forms a thin film adhering to its internal wall surface. The liquid stream 330 flowrate is such adjusted that a uniform liquid film forms, wets the internal wall surfaces and downflows in Unit 330. A heat input stream 399 is supplied to Unit 330 that provided the necessary energy for generating the vapors from the falling liquid film. The FFE Unit 330 may employ internally mounted rotating blades or wipers to aid in evenly spreading the falling liquid film over the wall surfaces.

The FFE Unit 330 vaporizes part of the liquid side-draw stream 315 and the up-flowing vapor stream 317 is returned to the column Unit 310 above the side-draw collection section or tray of Unit 310. The FFE Unit 330 bottoms stream may either be returned to the column 310 below the side-draw collection section or tray (stream 323) or simply taken out of the system (stream 319).

This example demonstrates how the FFE Unit 330 can be configured to operate on the liquid side-draw stream from the distillation unit 310. In doing so, the hold-up time for the materials exposed to the column 310 base conditions is reduced along with the reduced levels of zinc chloride concentration at the unit 310 base.

Reduced formation of CPI is observed that results in the reduced levels of CPI in the refined adiponitrile stream 309 overhead.

One or multiple FFE units, operating on one or more liquid side-draw streams from the distillation unit may be envisioned depending on the process throughput. Such multiple FFE units can be designed, sized and flow connected using the conventional means. These may be cascading, parallel, series or any combination of such FFEs that are easy to operate and maintain.

While the illustrative embodiments of the invention have been described with particularity, it will be understood that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present invention, including all features which would be treated as equivalents thereof by those skilled in the art to which the invention pertains.

1. A method for purifying a crude adiponitrile stream by differential volatility comprising separating at least a portion of the components of the crude adiponitrile stream by flashing vapor from a falling liquid film.

2. The method of claim 1 wherein the liquid film flows downwardly on a substantially vertical wall.

3. The method of claim 1 wherein the heat of vaporization for flashing vapor is at least partially sensible heat of the liquid film.

4. The method of claim 1 wherein CPI is a component of the crude adiponitrile stream.

5. The method of claim 1wherein the step of flashing vapor from a liquid film is followed by multistage distillation.

6. The method of claim 1wherein the step of flashing vapor from a liquid film is preceded by multistage distillation.

7. The method of claim 1wherein the step of flashing vapor from a liquid film is preceded by multistage distillation and followed by multistage distillation.

8. The method of claim 1 wherein the step of flashing vapor from a liquid film is preceded by multistage distillation and followed by multistage distillation, wherein the multistage distillation steps are carried out in a single distillation tower.

9. The method of claim 1 wherein the multistage distillation is carried out under at least partial vacuum.

10. The method of claim 6 wherein the step of flashing vapor from a liquid film is carried out under at least partial vacuum.

11. The method of claim 1 further comprising controlling the temperature of the liquid film to reduce formation of CPI.

12. The method of claim 1 wherein the flashing from the liquid film is carried out at conditions including:
a. Temperature of from 160° C. to 220° C.; and
b. Pressure of from 0.3 psia to 0.6 psia.

13. The method of claim 12 wherein the conditions include:
a. Temperature of from 175° C. to 205° C.; and
b. Pressure of from 0.35 psia to 0.5 psia.

14. The method of claim 13 wherein the conditions include:
a. Temperature of from 180° C. to 200° C.; and
b. Pressure of from 0.4 psia to 0.45 psia.

15. The method of claim 1 wherein from ≥0% to less than 20% by weight of liquid flashed to vapor is flashed from a horizontal surface.

16. The method of claim 15 wherein from ≥0% to ≤10% by weight of liquid flashed to vapor is flashed from a horizontal surface.

17. The method of claim 16 wherein from ≥0% to ≤5% by weight of liquid flashed to vapor is flashed from a horizontal surface.
